(19) United States
(12) Patent Application Publication
(10) Pub. No.: US 2026/0272941 A1
(43) Pub. Date: Sep. 17, 2026

(54) TREATMENT OF MALIGNANT TUMOR

(71) Applicant: The University of Tokyo, Tokyo (JP)

(72) Inventors: Takahisa MURATA, Tokyo (JP); Kanan YASUI, Tokyo (JP); Koji KOBAYASHI, Tokyo (JP)

(21) Appl. No.: 19/563,879

(22) Filed: Mar. 11, 2026

(30) Foreign Application Priority Data
Mar. 12, 2025 (JP) 2025-039793

(51) Int. Cl. A61K 31/5377; A61K 31/196; A61K 31/403; A61K 31/437; A61K 31/454; A61K 39/00; A61P 35/00; C07K 16/28

(52) U.S. Cl. CPC A61K 31/5377; A61K 31/196; A61K 31/403; A61K 31/437; A61K 31/454; A61P 35/00; C07K 16/2818; C07K 16/2827; A61K 2039/505

(57) ABSTRACT

[Problem] To provide a method for treating a malignant tumor.

[Solution] Provided is a method for treating a malignant tumor, comprising administering a PGD2-CRTH2 signal inhibitor and an immunotherapeutic agent to a subject. Alternatively, provided is a pharmaceutical composition for use in the treatment of a malignant tumor, wherein the treatment comprises a combination treatment using a PGD2-CRTH2 signal inhibitor and an immunotherapeutic agent for a subject, and the pharmaceutical composition comprises a PGD2-CRTH2 signal inhibitor or an immunotherapeutic agent.

### TECHNICAL FIELD

The technical field of the present invention relates to the treatment of malignant tumors.

### BACKGROUND ART

Malignant tumors are diseases that rank among the leading causes of death. However, the effects of existing therapeutic methods are limited, and there is a need for the development of novel therapeutic methods that have fewer side effects and high therapeutic efficacy. Immunotherapeutic agents that activate immune cells capable of recognizing tumor antigens and to attack and kill tumor cells have attracted attention in recent years. For example, an anti-PD-1 antibody, one of the immune checkpoint inhibitors, exerts an anti-tumor effect by inhibiting PD-L1 expressed on tumor cells from binding to the receptor PD-1 expressed on CD8-positive T cells, thereby reactivating the CD8-positive T cells whose anti-tumor immune function is suppressed (Non-Patent Literature 1). However, applicable cancer types are limited. Furthermore, even in applicable cancer types such as melanoma and breast cancer, patients in whom a therapeutic effect is observed are limited to approximately 20-30%. Therefore, prediction of efficacy by biomarkers and combination with other anti-cancer agents (Non-Patent Literature 2) have been studied.

### CITATION LIST

### Non-Patent Literature

Non-Patent Literature 1: Jedd D et al., Nature. 2014, 515(7528):496-8
Non-Patent Literature 2: Wensheng Chen et al., Biochem. Pharmacol., 2022, 202:115113

### SUMMARY OF INVENTION

### Technical Problem

Many points remained unclear regarding the therapeutic effects and treatment methods for malignant tumors using immunotherapeutic agents.

On the other hand, the present inventors have found that a combination treatment using a PGD2-CRTH2 signal inhibitor and an immunotherapeutic agent has an excellent therapeutic effect against malignant tumors.

### Solution to Problem

An aspect of the present invention provides a method for treating a malignant tumor, comprising administering a PGD2-CRTH2 signal inhibitor and an immunotherapeutic agent to a subject. By using this treatment method, a superior therapeutic effect against a malignant tumor can be obtained.

An aspect of the present invention provides a pharmaceutical composition for use in the treatment of a malignant tumor, wherein the treatment comprises a combination treatment using a PGD2-CRTH2 signal inhibitor and an immunotherapeutic agent for a subject, and the pharmaceutical composition comprises the PGD2-CRTH2 signal inhibitor or the immunotherapeutic agent. By using this pharmaceutical composition, a more excellent therapeutic effect against a malignant tumor can be obtained.

### BRIEF DESCRIPTION OF DRAWINGS

FIG. 1 shows tumor volume (mm³) in a skin cancer mouse model (wild-type (WT)) after administration of vehicle, an anti-PD-1 antibody, or a combination (anti-PD-1 antibody+ramatroban). N=7-12. Significant difference relative to the WT vehicle group: **P<0.01.

FIG. 2 shows tumor volume (mm³) in a skin cancer mouse model (wild-type (WT)) after administration of vehicle, an anti-PD-1 antibody, or a combination (anti-PD-1 antibody+setipiprant). N=7-12.

FIG. 3 shows tumor volume (mm³) in skin cancer mouse models (wild-type (WT) and CRTH2 KO) after administration of vehicle or an anti-PD-1 antibody. N=5-11. Significant difference relative to WT vehicle group: **P<0.01. Significant difference relative to the CRTH2−/− vehicle group: #P<0.05.

FIG. 4 shows typical H&E stained images of tumor tissues from skin cancer mouse models (wild-type (WT) and CRTH2 KO) after administration of vehicle or an anti-PD-1 antibody. Upper left: WT mouse vehicle group; lower left: WT mouse anti-PD-1 antibody-treated group (5 mg/kg); upper right: CRTH2−/− mouse vehicle group; lower right: CRTH2−/− mouse anti-PD-1 antibody-treated group (5 mg/kg). Scale bar=100 μm.

FIG. 5 shows the quantitative results of H&E staining of tumor tissues of skin cancer mouse models (wild-type (WT) and CRTH2 KO) after administration of vehicle or an anti-PD-1 antibody. N=5-7. Significant difference relative to the WT vehicle group: *P<0.05. Significant difference relative to the WT αPD-1 group: ††P<0.01. Significant difference relative to the CRTH2−/− vehicle group: #P<0.05.

FIG. 6 shows tumor volume (mm³) in skin cancer mouse models (wild-type (WT) and CRTH2 KO) after administration of vehicle or an anti-CTLA-4 antibody. N=2-4.

FIG. 7 shows tumor volume (mm³) in breast cancer mouse models (wild-type (WT) and CRTH2 KO) after administration of vehicle or an anti-PD-1 antibody. N=1-3.

FIG. 8 shows tumor volume (mm³) in a skin cancer mouse model (wild-type (WT)) after administration of vehicle, an anti-PD-1 antibody, or a combination (anti-PD-1 antibody+TFC-007). N=3-4.

FIG. 9 shows tumor volume (mm³) in skin cancer mouse models (wild-type (WT) and HPGDS KO) after administration of vehicle or an anti-PD-1 antibody. N=5-11. Significant difference relative to WT vehicle group: **P<0.01. Significant difference relative to the H-PGDS−/− vehicle group: #P<0.05.

### DESCRIPTION OF EMBODIMENTS

Hereinafter, embodiments of the present invention will be described in detail. Note that repeated descriptions of similar content are omitted where appropriate to avoid repetition.

### (1) Method

An embodiment of the present invention provides a method for treating a malignant tumor, comprising administering a PGD2-CRTH2 signal inhibitor to a subject. This method may be performed, for example, in a combination treatment using a PGD2-CRTH2 signal inhibitor and an immunotherapeutic agent. This method may comprise, for example, administering an immunotherapeutic agent to the subject. This method may comprise, for example, performing immunotherapy on the subject. By the combination treatment, a superior therapeutic effect against a malignant tumor can be obtained. The superior therapeutic effect may include, for example, a tumor growth inhibitory effect on a primary cancer, an inhibitory effect on metastasis or recurrence, or a survival-prolonging effect in the subject. The superior therapeutic effect may include, for example, a synergistic therapeutic effect by the combination treatment. This embodiment provides a method for treating a malignant tumor, comprising administering a PGD2-CRTH2 signal inhibitor to a subject who has undergone immunotherapy. This embodiment provides a method for treating a malignant tumor, comprising administering a PGD2-CRTH2 signal inhibitor and an immunotherapeutic agent to a subject. This embodiment provides a method for treating a malignant tumor, comprising administering a PGD2-CRTH2 signal inhibitor and an immunotherapeutic agent in combination to a subject.

### (2) Method

An embodiment of the present invention provides a method for treating a malignant tumor, comprising administering an immunotherapeutic agent to a subject. This method may be performed, for example, in a combination treatment using a PGD2-CRTH2 signal inhibitor and an immunotherapeutic agent. This method may comprise, for example, administering a PGD2-CRTH2 signal inhibitor to the subject. By the combination treatment, a superior therapeutic effect against a malignant tumor can be obtained. This embodiment provides a method for treating a malignant tumor, comprising administering an immunotherapeutic agent to a subject who has been administered a PGD2-CRTH2 signal inhibitor.

### (3) Method

An embodiment of the present invention provides a method for treating a malignant tumor, comprising combination treatment using administration of a PGD2-CRTH2 signal inhibitor and an immunotherapeutic agent. This method may comprise, for example, administering a PGD2-CRTH2 signal inhibitor to a subject, or administering an immunotherapeutic agent to a subject. By the combination treatment, a superior therapeutic effect against a malignant tumor can be obtained.

### (4) Method

The method of an embodiment of the present invention (e.g., (1) to (3) above) may comprise steps, for example, (i) testing for the presence or absence of a malignant tumor in a subject, (ii) identifying a subject having a malignant tumor, (iii) identifying a subject in need of treatment for a malignant tumor, or (iv) identifying a subject in need of prevention of a malignant tumor. Furthermore, the method of an embodiment of the present invention (e.g., (1) to (3) above) may comprise steps, for example, testing for expression of an immune checkpoint molecule or a ligand thereof (e.g., PD-L1) in a subject, detecting a tumor cell positive for an immune checkpoint molecule or a ligand thereof in a subject, identifying, as a treatment target, a subject in whom expression of an immune checkpoint molecule or a ligand thereof is confirmed. The method of an embodiment of the present invention (e.g., (1) to (3) above) may comprise steps, for example, testing for a PGD2 (a ligand of PGD2-CRTH2 signal) metabolite in a body fluid of a subject, identifying, as a treatment target, a subject in whom a PGD2 metabolite is detected in a body fluid.

### (5) Composition

An embodiment of the present invention provides a pharmaceutical composition for use in the treatment of a malignant tumor, wherein the pharmaceutical composition comprises a PGD2-CRTH2 signal inhibitor. This treatment may be, for example, a combination treatment using a PGD2-CRTH2 signal inhibitor and an immunotherapeutic agent for a subject. By the combination treatment, a superior therapeutic effect against a malignant tumor can be obtained. The superior therapeutic effect may include, for example, a tumor growth inhibitory effect on a primary cancer, an inhibitory effect on metastasis or recurrence, or a survival-prolonging effect for the subject. The superior therapeutic effect may include, for example, a synergistic therapeutic effect by the combination treatment.

### (6) Composition

An embodiment of the present invention provides a pharmaceutical composition for use in the treatment of a malignant tumor, wherein the pharmaceutical composition comprises an immunotherapeutic agent. This treatment may be, for example, a combination treatment using a PGD2-CRTH2 signal inhibitor and an immunotherapeutic agent for a subject. By the combination treatment, a superior therapeutic effect against a malignant tumor can be obtained.

### (7) Composition

An embodiment of the present invention provides a pharmaceutical composition for use in the treatment of a malignant tumor, comprising a PGD2-CRTH2 signal inhibitor and an immunotherapeutic agent. By this pharmaceutical composition, a superior therapeutic effect against a malignant tumor can be obtained.

### (8) Composition

An embodiment of the present invention provides a pharmaceutical composition for use in the treatment of a malignant tumor, wherein the treatment comprises a combination treatment using a PGD2-CRTH2 signal inhibitor and an immunotherapeutic agent for a subject. This pharmaceutical composition may comprise, for example, a PGD2-CRTH2 signal inhibitor or an immunotherapeutic agent. By the combination treatment, a superior therapeutic effect against a malignant tumor can be obtained. This embodiment provides a pharmaceutical composition for use in the treatment of a malignant tumor, wherein the treatment comprises a combination treatment using a PGD2-CRTH2 signal inhibitor and an immunotherapeutic agent for a subject, and the pharmaceutical composition comprises a PGD2-CRTH2 signal inhibitor or an immunotherapeutic agent.

### (9) Composition

An embodiment of the present invention provides a pharmaceutical composition comprising a PGD2-CRTH2 signal inhibitor for use in the methods described in (1) to (4) above. Another embodiment provides a pharmaceutical composition comprising an immunotherapeutic agent for use in the methods described in (1) to (4) above.

### (10) Kit

An embodiment of the present invention provides a kit comprising the pharmaceutical composition described in (5) to (9) above. The kit may comprise, for example, an instruction manual, a buffer, a container, or a package. Another embodiment provides a kit for use in the treatment of a malignant tumor, comprising a PGD2-CRTH2 signal inhibitor and an immunotherapeutic agent.

### (11) Use

An embodiment of the present invention provides a use of a PGD2-CRTH2 signal inhibitor for the manufacture of the pharmaceutical composition described in (5) to (9) above. Another embodiment provides a use of an immunotherapeutic agent in the manufacture of the pharmaceutical composition described in (5) to (9) above.

In one embodiment of the present invention (e.g., (1) to (11) above), the PGD2-CRTH2 signal refers to a signal pathway in which PGD2, one of the prostaglandins (PGs) that play a central role in inflammatory reactions, is produced from arachidonic acid as a substrate, in the cell membranes of immune cells by cyclooxygenase (COX) and PGD2 synthase (H-PGDS) or lipocalin-type PGD2 synthase (L-PGDS), and the produced PGD2 acts on CRTH2 (a G protein-coupled receptor) to exert physiological activity.

In one embodiment of the present invention (e.g., (1) to (11) above), the PGD2-CRTH2 signal inhibitor may be a CRTH2 inhibitor or a PGD2 synthase inhibitor. The PGD2 synthase inhibitor may be an H-PGDS inhibitor or an L-PGDS inhibitor. The form of the “inhibitor” is not particularly limited, and may be, for example, a low-molecular-weight compound (e.g., a low-molecular-weight organic compound), a polynucleotide (e.g., an RNA strand or a DNA strand), an antibody, or a polypeptide (e.g., an enzyme), or a composition containing the same. The low-molecular-weight compound may be obtained, for example, by an FDA library screening. Alternatively, it can be obtained by utilizing combinatorial chemistry or HTS (High-Throughput Screening). For the combinatorial chemistry, for example, an automatic synthesizer, the L-COS series (Shoko Scientific Co., Ltd.) may be used. For HTS, for example, an Octet system (ForteBio) may be used. Alternatively, the low-molecular-weight compound may be a PROTAC (Proteolysis-Targeting Chimera).

The CRTH2 inhibitor may be, for example, the following compounds, pharmaceutically acceptable salts thereof, or solvates thereof:
(1) Ramatroban ((+)-(3R)-3-[[(4-fluorophenyl)sulfonyl]amino]-1,2,3,4-tetrahydro-9H-carbazole-9-propanoic acid, CAS 116649-85-5);
(2) 4-(3-{3-[3-(diphenylmethyl)-6-oxopyridazin-1(6H)-yl]propyl}phenoxy)butanoic acid;
(3) 1-{6-[2-(2,4-dichloro-phenyl)-ethylamino]-2-methyl-pyrimidin-4-yl}-pyrrolidine-3-carboxylic acid;
(4) 2-(1-{2-methoxy-6-[2-(4-trifluoromethoxy-phenyl)-ethylamino]-pyrimidin-4-yl}-piperidin-3-yl)-2-methyl-propionic acid;
(5) 2-[3-{6-[2-(2,4-dichloro-phenyl)-ethylamino]-2-methoxy-pyrimidin-4-yl}-5-(1-hydroxy-1-methyl-ethyl)-phenyl]-propan-2-ol;
(6) [6-(3-amino-piperidin-1-yl)-2-methoxy-pyrimidin-4-yl]-[2-(2,4-dichloro-phenyl)-ethyl]-amine;
(7) [6-(4-amino-piperidin-1-yl)-2-methoxy-pyrimidin-4-yl]-[2-(2,4-dichloro-phenyl)-ethyl]-amine;
(8) N-(1-{6-[2-(2,4-dichloro-phenyl)-ethylamino]-2-methoxy-pyrimidin-4-yl}-piperidin-4-yl)-acetamide;
(9) 5-{6-[2-(2,4-dichloro-phenyl)-ethylamino]-2-methoxy-pyrimidin-4-yl}-1-methyl-2,3-dihydro-1H-indole-2-carboxylic acid;
(10) 2-methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide (11) N,N-dimethylamido-2-sulfonic acid [2-(3-{6-[2-(2,4-dichloro-phenyl)-ethylamino]-2-methoxy-pyrimidin-4-yl}-phenyl)-2-methyl-propionyl]-amide;
(12) 2-(3-{6-[2-(2,4-dichloro-phenyl)-ethylamino]-2-methoxy-pyrimidin-4-yl}-phenyl)-2-methyl-1-thiomorpholin-4-yl-propan-1-one;
(13) 2-(3-{6-[2-(2,4-dichloro-phenyl)-ethylamino]-2-methoxy-pyrimidin-4-yl}-phenyl)-isobutyramide;
(14) 2-(3-{6-[2-(2,4-dichloro-phenyl)-ethylamino]-2-methoxy-pyrimidin-4-yl}-phenyl)-N,N-dimethyl-isobutyramide;
(15) (1-{6-[2-(2,4-dichloro-phenyl)-ethylamino]-2-methoxy-pyrimidin-4-yl}-piperidin-3-yl)-acetic acid;
(16) 1-{2-methoxy-6-[2-(4-trifluoromethoxy-phenyl)-ethylamino]-pyrimidin-4-yl}-piperidine-3-carboxylic acid;
(17) N-(1-{2-methoxy-6-[2-(4-trifluoromethoxy-phenyl)-ethylamino]-pyrimidin-4-yl}-piperidine-3-carbonyl)-methanesulfonamide;
(18) 5-{6-[2-(2,4-dichloro-phenyl)-ethylamino]-2-methoxy-pyrimidin-4-yl}-1-methyl-2,3-dihydro-1H-indole-2-carboxylic acid ethyl ester;
(19) (1-{6-[2-(2,4-dichloro-phenyl)-ethylamino]-2-methoxy-pyrimidin-4-yl}-piperidin-3-yl)-acetic acid ethyl ester;
(20) N-(1-{6-[2-(2,4-dichloro-phenyl)-ethylamino]-2-methoxy-pyrimidin-4-yl}-piperidine-3-carbonyl)-methanesulfonamide;
(21) Ethanesulfonic acid (1-{6-[2-(2,4-dichloro-phenyl)-ethylamino]-2-methoxy-pyrimidin-4-yl}-piperidine-3-carbonyl)-amide;
(22) 2-methyl-propane-2-sulfonic acid (1-{6-[2-(2,4-dichloro-phenyl)-ethylamino]-2-methoxy-pyrimidin-4-yl}-piperidine-3-carbonyl)-amide;
(23) N-(1-{6-[2-(2,4-dichloro-phenyl)-ethylamino]-2-methoxy-pyrimidin-4-yl}-piperidine-3-carbonyl)-C, C, C-trifluoro-methanesulfonamide;
(24) 1-{6-[2-(2,4-dichloro-phenyl)-ethylamino]-2-methoxy-pyrimidin-4-yl}-piperidine-3-carboxylic acid (1H-tetrazol-5-yl)-amide;
(25) 1-{6-[2-(2,4-dichloro-phenyl)-ethylamino]-2-methoxy-pyrimidin-4-yl}-piperidine-3-carboxylic acid amide;
(26) 1-{6-[2-(2,4-dichloro-phenyl)-ethylamino]-2-methoxy-pyrimidin-4-yl}-piperidine-3-carboxylic acid dimethylamide;
(27) N,N-dimethylamido-2-sulfonic acid 1-{6-[2-(2,4-dichloro-phenyl)-ethylamino]-2-methoxy-pyrimidin-4-yl}-piperidine-3-carboxyamide;
(28) 5-{2-methoxy-6-[2-(4-trifluoromethoxy-phenyl)-ethylamino]-pyrimidin-4-yl}-thiophene-2-carboxylic acid;
(29) 5-{6-[2-(2,4-dichloro-phenyl)-ethylamino]-2-methoxy-pyrimidin-4-yl}-2,3-dihydro-benzofuran-2-carboxylic acid;
(30) 2-(3-{6-[2-(2,4-dichloro-phenyl)-ethylamino]-2-methoxy-pyrimidin-4-yl}-phenyl)-2-methyl-propionic acid;
(31) 4-{cyclopropyl[cis,cis-4-{[4-(trifluoromethoxy)phenyl]carbonyl}-2,3,3a,4,9,9a-hexahydro-1H-cyclopenta[b]quinolin-9-yl]amino}-4-oxobutanoic acid;
(32) 4-[cyclopropyl[cis,cis-7-fluoro-2,3,3a,4,9,9a-hexahydro-4-[4-(trifluoromethoxy)benzoyl]-1H-cyclopenta[b]quinolin-9-yl]amino]-4-oxobutanoic acid;
(33) 4-[[cis,cis-6-chloro-7-fluoro-2,3,3a,4,9,9a-hexahydro-4-[4-(trifluoromethoxy)benzoyl]-1H-cyclopenta[b]quinolin-9-yl]cyclopropylamino]-4-oxobutanoic acid;
(34) 4-[cyclopropyl[cis,cis-5-fluoro-1,2,2a,3,8,8a-hexahydro-3-[4-(trifluoromethoxy)benzoyl]cyclobuta[b]quinolin-8-yl]amino]-4-oxobutanoic acid;
(35) 4-[cyclopropyl[cis,cis-6-fluoro-1,2,2a,3,8,8a-hexahydro-3-[4-(trifluoromethoxy)benzoyl]cyclobuta[b]quinolin-8-yl]amino]-4-oxobutanoic acid;
(36) (cis,cis)-8-[(3-carboxy-1-oxopropyl)cyclopropylamino]-5-chloro-2,2a,8,8a-tetrahydrocyclobuta[b]quinoline-3(1H)-carboxylic acid 3-(phenylmethyl);
(37) (cis,cis)-8-[(3-carboxy-1-oxopropyl)cyclopropylamino]-2,2a,8,8a-tetrahydrocyclobuta[b]quinoline-3(1H)-carboxylic acid 3-(phenylmethyl);
(38) 4-[cyclopropyl[cis,cis-1,2,2a,3,8,8a-hexahydro-3-[4-(trifluoromethoxy)benzoyl]cyclobuta[b]quinolin-8-yl]amino]-4-oxobutanoic acid;
(39) [[[cyclopropyl[cis,cis-3-(3,4-difluorobenzoyl)-1,2,2a,3,8,8a-hexahydrocyclobuta[b]quinolin-8-yl]amino]carbonyl]oxy]acetic acid;
(40) cis,cis-8-[(3-carboxypropyl)cyclopropylamino]-5-fluoro-2,2a,8,8a-tetrahydrocyclobuta[b]quinoline-3(1H)-carboxylic acid 3-(phenylmethyl);
(41) 4-[cyclopropyl[cis,cis-2,3,3a,4,9,9a-hexahydro-4-[4-(trifluoromethoxy)benzoyl]-1H-cyclopenta[b]quinolin-9-yl]amino]butanoic acid;
(42) 4-[cyclopropyl[cis,cis-2,3,3a,4,9,9a-hexahydro-4-[4-[(trifluoromethyl)thio]benzoyl]-1H-cyclopenta[b]quinolin-9-yl]amino]-4-oxobutanoic acid;
(43) (R)-1-((cis,cis-3-(benzyloxycarbonyl)-5,6-difluoro-1,2,2a,3,8,8a-hexahydrocyclobuta[b]quinolin-8-yl)(cyclopropyl)carbamoyl)azetidine-2-carboxylic acid;
(44) 4-(cyclopropyl(cis,cis-3-(4-((trifluoromethyl)thio)benzoyl)-1,2,2a,3,8,8a-hexahydrocyclobuta[b]quinolin-8-yl)amino)-4-oxobutanoic acid;
(45) 4-(ethyl(cis,cis-6-fluoro-3-(4-((trifluoromethyl)thio)benzoyl)-1,2,2a,3,8,8a-hexahydrocyclobuta[b]quinolin-8-yl)amino)-4-oxobutanoic acid;
(46) 4-(ethyl(cis,cis-7-fluoro-4-(4-((trifluoromethyl)thio)benzoyl)-2,3,3a,4,9,9a-hexahydro-1H-cyclopenta[b]quinolin-9-yl)amino)-4-oxobutanoic acid;
(47) 4-(cyclopropyl(cis,cis-7-fluoro-4-(4-((trifluoromethyl)thio)benzoyl)-2,3,3a,4,9,9a-hexahydro-1H-cyclopenta[b]quinolin-9-yl)amino)-4-oxobutanoic acid;
(48) 4-(cyclopropyl(cis,cis-7-fluoro-4-(4-(trifluoromethoxy)benzoyl)-2,3,3a,4,9,9a-hexahydro-1H-cyclopenta[b]quinolin-9-yl)amino)butanoic acid;
(49) {3-[1-(4-chloro-phenyl)-ethyl]-5-fluoro-2-methyl-indol-1-yl}-acetic acid;
(50) {5-fluoro-2-methyl-3-[1-(4-trifluoromethyl-phenyl)-ethyl]-indol-1-yl}-acetic acid;
(51) {3-[1-(4-tert-butyl-phenyl)-ethyl]-5-fluoro-2-methyl-indol-1-yl}-acetic acid;
(52) {5-fluoro-3-[1-(4-methanesulfonyl-phenyl)-ethyl]-2-methyl-indol-1-yl}-acetic acid;
(53) [5-fluoro-2-methyl-3-(1-naphthalen-2-yl-ethyl)-indol-1-yl]-acetic acid;
(54) (5-fluoro-2-methyl-3-quinolin-2-ylmethyl-indol-1-yl)-acetic acid;
(55) (5-fluoro-2-methyl-3-naphthalen-2-ylmethyl-indol-1-yl)-acetic acid;
(56) [5-fluoro-3-(8-hydroxy-quinolin-2-ylmethyl)-2-methyl-indol-1-yl]-acetic acid;
(57) (5-fluoro-2-methyl-3-quinoxalin-2-ylmethyl-indol-1-yl)-acetic acid;
(58) [5-fluoro-3-(4-methoxy-benzyl)-2-methyl-indol-1-yl]-acetic acid;
(59) (5-fluoro-2-methyl-3-thiazol-2-ylmethyl-indol-1-yl)-acetic acid ethyl ester;
(60) [3-(4-chloro-benzyl)-5-fluoro-2-methyl-indol-1-yl]-acetic acid;
(61) [5-fluoro-2-methyl-3-(4-trifluoromethyl-benzyl)-indol-1-yl]-acetic acid;
(62) [5-fluoro-2-methyl-3-(4-tert-butyl-benzyl)-indol-1-yl]-acetic acid;
(63) (3-biphenyl-4-ylmethyl-5-fluoro-2-methyl-indol-1-yl)-acetic acid;
(64) [5-fluoro-3-(4-methanesulfonyl-benzyl)-2-methyl-indol-1-yl]-acetic acid;
(65) [5-fluoro-3-(6-fluoro-quinolin-2-ylmethyl)-2-methyl-indol-1-yl]-acetic acid;
(66) (2-methyl-3-quinolin-2-ylmethyl-indol-1-yl)-acetic acid;
(67) (5-chloro-2-methyl-3-quinolin-2-ylmethyl-indol-1-yl)-acetic acid;
(68) {2-[4-chloro-3-(2,6-dichloro-benzylsulfamoyl)-phenyl]-1H-indol-3-yl}-acetic acid;
(69) {2-[4-chloro-3-(3,5-dichloro-benzylsulfamoyl)-phenyl]-1H-indol-3-yl}-acetic acid;
(70) (2-{4-chloro-3-[2-(2,4-dichloro-phenyl)-ethylsulfamoyl]-phenyl}-1H-indol-3-yl)-acetic acid;
(71) (2-{4-chloro-3-[2-(2-methoxy-phenyl)-ethylsulfamoyl]-phenyl}-1H-indol-3-yl)-acetic acid;
(72) (2-{4-chloro-3-[2-(3-methoxy-phenyl)-ethylsulfamoyl]-phenyl}-1H-indol-3-yl)-acetic acid;
(73) (2-{4-chloro-3-[2-(4-methoxy-phenyl)-ethylsulfamoyl]-phenyl}-1H-indol-3-yl)-acetic acid;
(74) (2-{4-chloro-3-[2-(2-trifluoromethoxy-phenyl)-ethylsulfamoyl]-phenyl}-1H-indol-3-yl)-acetic acid;
(75) [2-(4-chloro-3-phenethylsulfamoyl-phenyl)-1H-indol-3-yl]-acetic acid;
(76) {2-[4-chloro-3-(3-phenyl-propylsulfamoyl)-phenyl]-1H-indol-3-yl}-acetic acid; (77) 2-[2-(4-chloro-3-cyclohexylsulfamoyl-phenyl)-1H-indol-3-yl]-N-methyl-acetamide;
(78) [4-chloro-2-(4-chloro-3-cyclohexylsulfamoyl-phenyl)-1H-indol-3-yl]-acetic acid;
(79) [4-chloro-2-(4-chloro-3-cyclohexylsulfamoyl-phenyl)-1H-indol-3-yl]-acetic acid potassium;
(80) [2-(4-chloro-3-cyclohexylsulfamoyl-phenyl)-4-fluoro-1H-indol-3-yl]-acetic acid;
(81) [2-(4-chloro-3-cyclohexylsulfamoyl-phenyl)-4-methyl-1H-indol-3-yl]-acetic acid;
(82) [7-chloro-2-(4-chloro-3-cyclohexylsulfamoyl-phenyl)-1H-indol-3-yl]-acetic acid;
(83) 2-chloro-N-cyclohexyl-5-[3-(2-methanesulfonylamino-2-oxo-ethyl)-1H-indol-2-yl]-benzenesulfonamide;
(84) 2-chloro-N-cyclohexyl-5-[3-(2-ethanesulfonylamino-2-oxo-ethyl)-1H-indol-2-yl]-benzenesulfonamide;
(85) 2-chloro-N-cyclohexyl-5-[3-(2-oxo-2-trifluoromethanesulfonylamino-ethyl)-1H-indol-2-yl]-benzenesulfonamide;
(86) 2-[2-(4-chloro-3-cyclohexylsulfamoyl-phenyl)-1H-indol-3-yl]-N-(1H-tetrazol-5-yl)-acetamide;
(87) [2-(3-cyclohexylsulfamoyl-4-ethyl-phenyl)-1H-indol-3-yl]-acetic acid;
(88) 2-[2-(4-chloro-3-cyclohexylsulfamoyl-phenyl)-1H-indol-3-yl]-propionic acid;
(89) {2-[4-chloro-3-(3-chloro-phenylmethanesulfonyl)-phenyl]-1H-indol-3-yl}-acetic acid; and (90) {2-[4-chloro-3-(3-chloro-phenylmethanesulfonylamino)-phenyl]-1H-indol-3-yl}-acetic acid;

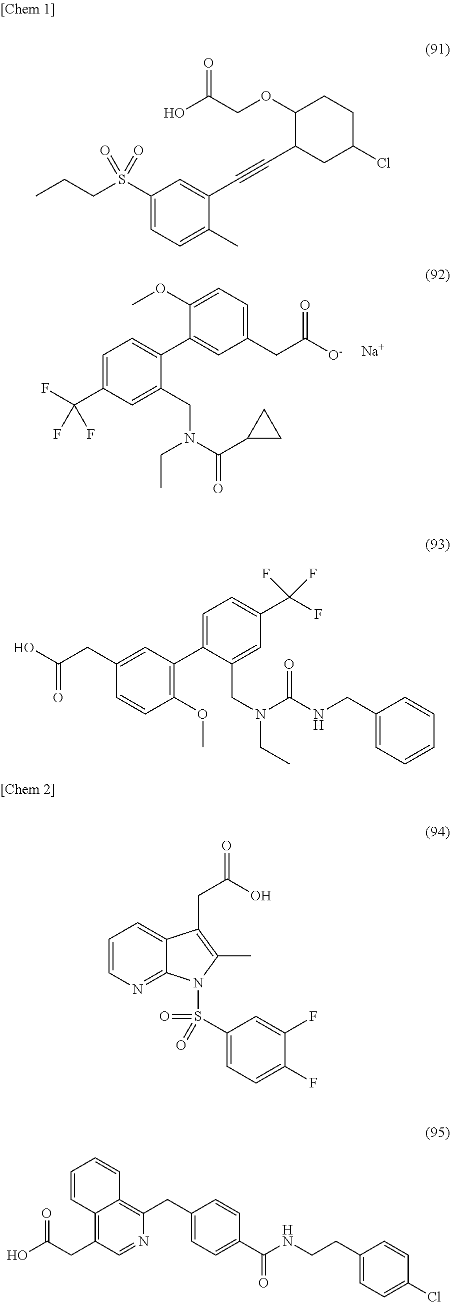 [SMILES: CCCS(=O)(=O)c1ccc(C)c(C#CC2CC(Cl)CCC2OCC(=O)O)c1 ; CCN(Cc1cc(C(F)(F)F)ccc1-c1cc(CC(=O)[O-])ccc1OC)C(=O)C1CC1 ; [Na+] ; CCN(Cc1cc(C(F)(F)F)ccc1-c1cc(CC(=O)O)ccc1OC)C(=O)NCc1ccccc1 ; Cc1c(CC(=O)O)c2cccnc2n1S(=O)(=O)c1ccc(F)c(F)c1 ; O=C(O)Cc1cnc(Cc2ccc(C(=O)NCCc3ccc(Cl)cc3)cc2)c2ccccc12]

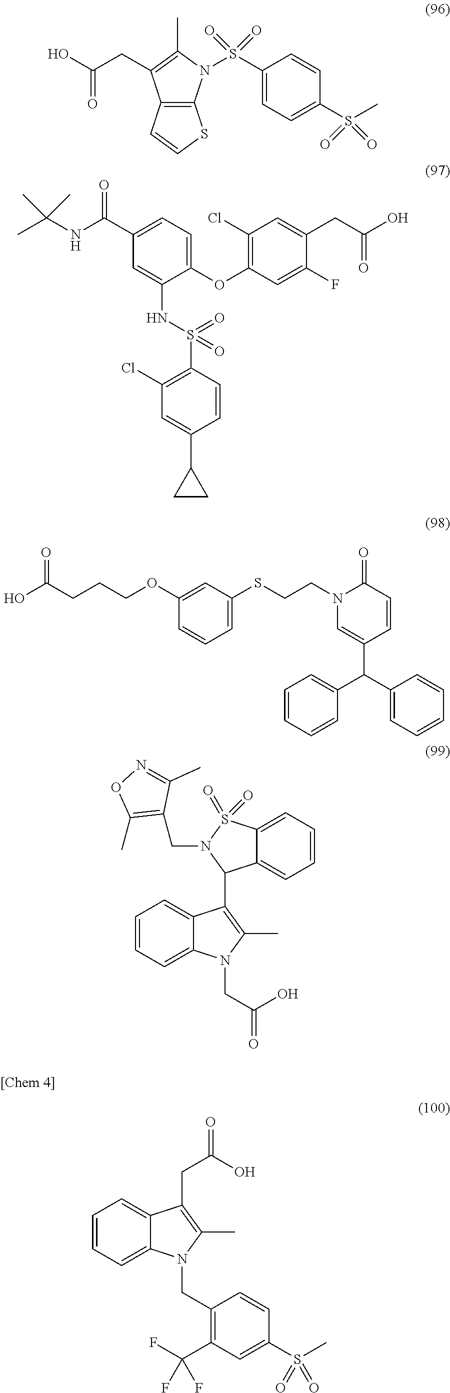

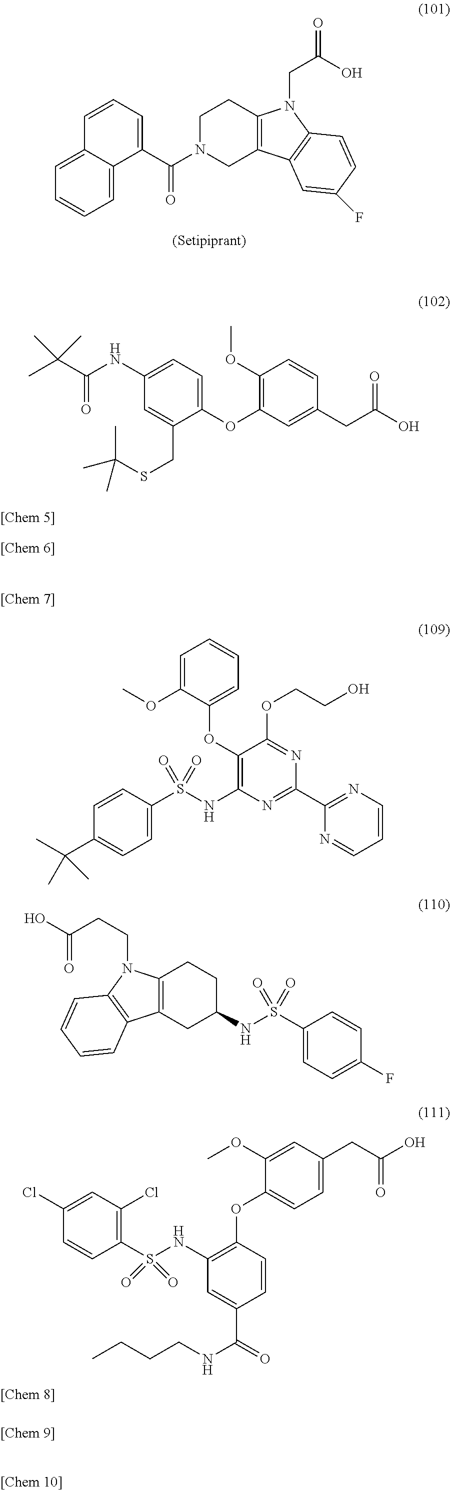

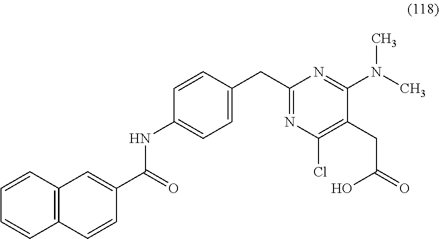 [SMILES: CN(C)c1nc(Cc2ccc(NC(=O)c3ccc4ccccc4c3)cc2)nc(Cl)c1CC(=O)O]

Alternatively, the CRTH2 inhibitor may be an antibody against CRTH2 (anti-CRTH2 antibody) or a PROTAC, or an siRNA against CRTH2 mRNA.

The H-PGDS inhibitor (hematopoietic prostaglandin D synthase inhibitor) may be TFC-007 (GAS No.: 927878-49-7), HQL-79 (GAS No.: 162641-16-9), tranilast (GAS No.: 53902-12-8), pizuglanstat (GAS No.: 1244967-98-3), or a pharmaceutically acceptable salt thereof, or a solvate thereof. Alternatively, the H-PGDS inhibitor may be an antibody against H-PGDS (anti-H-PGDS antibody) or a PROTAC, or an siRNA against H-PGDS mRNA.

The L-PGDS inhibitor may be AT-56 (GAS No.: 162640-98-4), or a pharmaceutically acceptable salt thereof, or a solvate thereof. Alternatively, the L-PGDS inhibitor may be an antibody against L-PGDS (anti-L-PGDS antibody) or a PROTAC, or an siRNA against L-PGDS mRNA.

In one embodiment of the present invention (e.g., (1) to (11) above), from the viewpoint of more effectively suppressing the growth of malignant tumor cells, preferred examples of the low-molecular-weight compound include: ramatroban or setipiprant, a pharmaceutically acceptable salt thereof, or a solvate thereof, as the CRTH2 inhibitor; and TFC-007, a pharmaceutically acceptable salt thereof, or a solvate thereof, as the PGD2 synthase inhibitor.

Ramatroban includes a compound having a CRTH2 antagonistic activity and having a structure represented by the following formula. Ramatroban can also be represented by the IUPAC name 3-[(3R)-3-[(4-fluorophenyl)sulfonylamino]-1,2,3,4-tetrahydrocarbazol-9-yl]propanoic acid. Ramatroban includes a compound represented by the CAS registry number 116649-85-5. Examples of prescription drugs containing ramatroban include Ramatroban Tablets 50 mg “KO” and Ramatroban Tablets 75 mg “KO”. Ramatroban is usually orally administered to an adult in a dose of 75 mg as ramatroban twice a day, after breakfast and after dinner (or before bedtime).

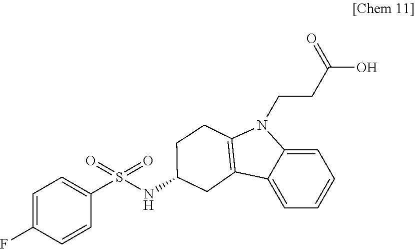 [SMILES: O=C(O)CCn1c2c(c3ccccc31)C[C@H](NS(=O)(=O)c1ccc(F)cc1)CC2]

Setipiprant includes a compound having a CRTH2 antagonistic activity and having a structure represented by the following formula. Setipiprant can also be represented by the IUPAC name 2-(2-(1-naphthoyl)-8-fluoro-1,2,3,4-tetrahydropyrido[4,3-b]indol-5-yl)acetic acid. Setipiprant includes a compound represented by the CAS registry number 866460-33-5.

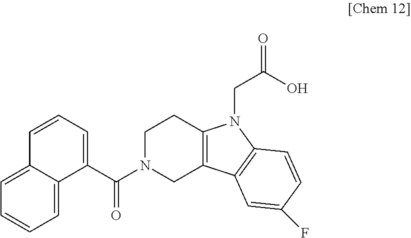 [SMILES: O=C(O)Cn1c2c(c3cc(F)ccc31)CN(C(=O)c1cccc3ccccc13)CC2]

TFC-007 is an inhibitor of H-PGDS, which is one of the PGD2 synthase inhibitors, and includes a compound having a structure represented by the following formula. TFC-007 can also be represented by the IUPAC name N-[4-[4-(4-morpholinylcarbonyl)-1-piperidinyl]phenyl]-2-phenoxy-5-pyrimidinecarboxamide. TFC-007 includes a compound represented by the CAS registry number 927878-49-7.

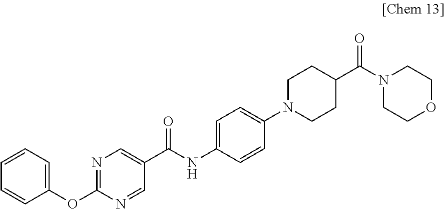 [SMILES: O=C(Nc1ccc(N2CCC(C(=O)N3CCOCC3)CC2)cc1)c1cnc(Oc2ccccc2)nc1]

In one embodiment of the present invention (e.g., (1) to (11) above), “combination” includes, performing another treatment before, simultaneously with, or after one treatment, for example, when two or more treatments (e.g., administrations) are present. Treatment before or after another treatment may be performed sequentially. Sequential performance includes, for example, performing one treatment for a certain period and then performing another treatment for a certain period in one treatment plan. Simultaneous treatment includes, for example, administering one formulation comprising two or more active ingredients to a subject. Combination treatment includes, for example, performing two or more treatment methods. In combination treatment, two or more treatment methods may be performed within one treatment plan. Combination treatment using a PGD2-CRTH2 signal inhibitor and an immunotherapeutic agent includes an aspect in which the administration of the former is performed before, simultaneously with, or after the administration of the latter.

In one embodiment of the present invention (e.g, (1) to (11) above), the administration of the PGD2-CRTH2 signal inhibitor may be performed before, simultaneously with, or after the administration of the immunotherapeutic agent. For example, administering a PGD2-CRTH2 signal inhibitor and an immunotherapeutic agent to a subject includes an aspect in which the administration of the PGD2-CRTH2 signal inhibitor is performed before, simultaneously with, or after the administration of the immunotherapeutic agent. The administration of the PGD2-CRTH2 signal inhibitor and the administration of the immunotherapeutic agent may be performed within one treatment plan.

In the method according to one embodiment of the present invention (e.g., (1) to (11) above), the step of identifying a subject may include a step of identifying a subject as a target for treatment or a step of identifying a subject as a target for administration of the pharmaceutical composition. The step of identifying a subject may include a step of selecting a subject or a step of identifying a subject.

In one embodiment of the present invention (e.g., (1) to (11) above), treatment includes being able to exert an action that produces a beneficial result for a patient (e.g., amelioration, mitigation, alleviation, cure, remission, or suppression (e.g., suppression of onset (e.g., prevention), suppression of progression, or suppression of recurrence)) with respect to the patient's disease (e.g., a malignant tumor) or one or more symptoms associated with the disease. The treatment or method may be performed by administering an effective amount (e.g., a therapeutically effective amount) of a PGD2-CRTH2 signal inhibitor to a subject. In one embodiment of the present invention (e.g., (1) to (11) above), the pharmaceutical composition may be produced by any method known in the technical field of pharmaceutics, for example, by mixing an active ingredient with one or more pharmaceutically acceptable carriers. The pharmaceutical composition is not limited in its form of use as long as it is used for treatment, and may be the active ingredient alone or a mixture of the active ingredient and any optional ingredients. Furthermore, the form of the carrier is not particularly limited, and may be, for example, a solid or a liquid (e.g., a buffer). The content of the carrier may be, for example, a pharmaceutically effective amount. The pharmaceutically effective amount may be, for example, an amount sufficient for the pharmaceutical stability or delivery of the active ingredient. The dosage may be, for example, 0.01 to 200 mg/kg body weight per dose. The pharmaceutical composition may contain a stabilizer, a buffer, or a pH adjuster. The dosage, administration interval, administration method, and administration route are not particularly limited and may be selected as appropriate depending on the age, body weight, symptoms, target organ, and the like of the patient. Furthermore, the pharmaceutical composition preferably contains a therapeutically effective amount, or an effective amount of an active ingredient to exert a desired effect. In one embodiment of the present invention, an effective amount includes an amount necessary for achieving a clinically observable improvement of symptoms in a patient. In one embodiment of the present invention, “pharmaceutically acceptable” includes a state suitable for use that is within the scope of sound medical judgment and is commensurate with a reasonable benefit/risk ratio. Ingredients other than the PGD2-CRTH2 signal inhibitor in the pharmaceutical composition are not particularly limited as long as they do not impair the effects of the present invention, and may be selected as appropriate depending on the purpose.

In one embodiment of the present invention (e.g., (1) to (11) above), the subject (including a patient) may be, for example, a subject in need of treatment for a malignant tumor. The subject in need of treatment for a malignant tumor may include, for example, a subject diagnosed as having a malignant tumor or a subject suspected of having a malignant tumor. The subject may be, for example, a subject in whom a malignant tumor marker is detected in a sample, or a subject in whom a malignant tumor marker in a sample is increased compared to a comparison subject (e.g., a healthy person). The subject may be, for example, a subject who has experienced suffering from a malignant tumor. The subject may be, for example, a subject undergoing treatment with a PGD2-CRTH2 signal inhibitor or a subject undergoing treatment with an immunotherapeutic agent. The subject may be, for example, a subject to whom a PGD2-CRTH2 signal inhibitor has been administered or a subject to whom an immunotherapeutic agent has been administered. The subject may be, for example, a subject who has a history of administration of a PGD2-CRTH2 signal inhibitor or a subject who has a history of administration of an immunotherapeutic agent. The subject may be, for example, a subject in whom leukopenia, anemia, or thrombocytopenia is observed. The subject may be, for example, a subject who has a decreased white blood cell count, is anemic, or has a decreased platelet count. The subject may be, for example, a subject in whom expression of an immune checkpoint molecule or a ligand thereof (e.g., PD-L1) is confirmed, a subject positive for an immune checkpoint molecule or a ligand thereof, a subject having tumor cells expressing an immune checkpoint molecule or a ligand thereof, or a subject having tumor cells positive for an immune checkpoint molecule or a ligand thereof. “Positive” may be, for example, a state of TPS (Tumor Proportion Score) 21%, ≥50%, or ≥1 to 50%. “Positive” may be, for example, a state of CPS (Combined Positive Score)≥1, ≥10, ≥20, or ≥1 to 20%. The subject includes a human or a non-human mammal (e.g., one or more of a mouse, guinea pig, hamster, rat, rabbit, pig, sheep, goat, cow, horse, cat, dog, marmoset, monkey, or chimpanzee).

In one embodiment of the present invention (e.g., (1) to (11) above), a malignant tumor includes, for example, a tumor that arises when a normal cell undergoes a gene mutation. A malignant tumor may arise from any organ or tissue throughout the body. A malignant tumor may include, for example, a solid tumor or a blood cancer. Solid tumors include, for example, one or more selected from the group consisting of lung cancer, uterine sarcoma, prostate cancer, head and neck cancer, colorectal cancer, gastric cancer, breast cancer, skin cancer, bladder cancer, kidney cancer, liver cancer, uterine cancer, pancreatic cancer, giant cell tumor of bone, ovarian cancer, esophageal cancer, adrenal cancer, biliary tract cancer, small intestinal cancer, ureteral cancer, renal pelvic cancer, penile cancer, testicular cancer, brain tumor (e.g., glioma), cancer of the central nervous system, cancer of the peripheral nervous system, thyroid cancer, salivary gland cancer, and carcinoma. The blood cancer includes, for example, lymphoma, leukemia, or multiple myeloma. The therapeutic effect on a malignant tumor may be evaluated, for example, by observing a change over time in the proliferation of malignant tumor cells after drug administration. The therapeutic effect on a malignant tumor may be evaluated, for example, by observing a reduction in tumor size or tumor volume after drug administration. In this case, when the tumor size or tumor burden significantly reduced compared to that before drug administration or upon administration of a negative control, a therapeutic effect may be considered present.

In one embodiment of the present invention (e.g., (1) to (11) above), immunotherapy includes a treatment method for attacking a malignant tumor by utilizing the immune function of a subject. For example, by inhibiting the function of an immune checkpoint molecule having an immunosuppressive effect, it is possible to release the immunosuppression and treat a malignant tumor. Immunotherapy includes, for example, administering an immunotherapeutic agent to a subject. In one embodiment of the present invention (e.g., (1) to (11) above), the immunotherapeutic agent includes, for example, an immune checkpoint inhibitor. The immune checkpoint molecule includes, for example, PD-1, CTLA-4, or LAG-3. The UniProt Primary accession numbers of these molecules can be identified by Q15116, P16410, and P18627, respectively. PD-1 can also be referred to as Programmed Cell Death 1. CTLA-4 can also be referred to as Cytotoxic T Lymphocyte Antigen-4. LAG-3 can also be referred to as Lymphocyte Activation Gene-3. Ligands of immune checkpoint molecules include, for example, PD-L1. PD-L1 can also be referred to as Programmed Cell Death Ligand 1. For the UniProt primary accession number of this molecule, Q9NZQ7 can be referred to. The immune checkpoint inhibitor may be, for example, an antibody that specifically binds to an immune checkpoint molecule or a ligand thereof. The antibody may be, for example, an antibody that inhibits the function of an immune checkpoint molecule or a ligand thereof. The antibody may be, for example, a monoclonal antibody. The antibody that specifically binds to an immune checkpoint molecule or a ligand thereof includes, for example, an anti-PD-1 antibody (e.g., nivolumab or pembrolizumab), an anti-PD-L1 antibody (e.g., atezolizumab or durvalumab), an anti-CTLA-4 antibody (e.g., ipilimumab), or an anti-LAG-3 antibody (e.g., relatlimab). The immunotherapeutic agent includes, for example, one or more agents selected from the group consisting of nivolumab, pembrolizumab, atezolizumab, durvalumab, ipilimumab, and relatlimab. For the CAS registry numbers of these molecules, 946414-94-4, 1374853-91-4, 1380723-44-3, 1428935-60-7, 477202-00-9, and 1673516-98-7 can be referred to, respectively. The immunotherapeutic agent may be, for example, an antibody having an amino acid sequence (e.g., heavy chain CDRs 1 to 3 and light chain CDRs 1 to 3) contained in these molecules. The amino acid sequence may be, for example, an amino acid sequence of a heavy chain, a light chain, a heavy chain variable region, a light chain variable region, heavy chain CDRs 1 to 3, or light chain CDRs 1 to 3. The immune checkpoint inhibitor includes, for example, a PD-1 inhibitor, a PD-L1 inhibitor, a CTLA-4 inhibitor, or a LAG-3 inhibitor. The immune checkpoint molecule or antibody may be of human origin.

In one embodiment of the present invention (e.g., (1) to (11) above), the salt is not particularly limited and includes, for example, an inorganic salt or an organic salt (see, e.g., Bharate et al., Drug Discov Today. 2021 February; 26(2):384-398. or Berge et al., J Pharm Sci. 1977 January; 66(1):1-19.). The salt includes, for example, a metal salt, an ammonium salt, a salt with an organic base, a salt with an inorganic acid, a salt with an organic acid, a salt with a basic or acidic amino acid, and the like. Metal salts include, for example, alkali metal salts (sodium salts, potassium salts, etc.), alkaline earth metal salts (calcium salts, magnesium salts, barium salts, etc.), aluminum salts, and the like. Salts with organic bases include, for example, salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, and the like. Salts with inorganic acids include, for example, salts with hydrochloric acid, hydrobromic acid, nitric acid, sulfuric acid, phosphoric acid, and the like. Salts with organic acids include, for example, salts with formic acid, acetic acid, trifluoroacetic acid, phthalic acid, fumaric acid, mesylic acid, tosylic acid, oxalic acid, tartaric acid, maleic acid, citric acid, succinic acid, malic acid, methanesulfonic acid, benzenesulfonic acid, p-toluenesulfonic acid, and the like. Salts with basic amino acids include, for example, salts with arginine, lysine, ornithine, and the like. Salts with acidic amino acids include, for example, salts with aspartic acid, glutamic acid, and the like. The salt includes a pharmaceutically acceptable salt. In one embodiment of the present invention, “pharmaceutically acceptable” includes a form that has a reasonable benefit for pharmaceutical use. In one embodiment of the present invention, one form of a compound or a salt thereof includes a form of a solvate thereof. In one embodiment of the present invention (e.g., (1) to (11) above), a solvate includes a form of a compound formed by a solute and a solvent (see, e.g., Healy et al., Adv Drug Deliv Rev. 2017 Aug. 1; 117:25-46.). The solvate includes, but is not particularly limited to, for example, a hydrate (e.g., a monohydrate, dihydrate, trihydrate, etc.) or a solvate with an organic solvent (e.g., a solvate with an alcohol (methanol, ethanol, propanol, etc.), acetone, dimethylformamide, or ethyl acetate, etc.). The solvent includes a solvent that can substantially maintain the physiological activity of a solute after forming a solvate. The solvate includes a pharmaceutically acceptable solvate.

In one embodiment of the present invention (e.g., (1) to (11) above), “significantly” may refer to, for example, a state in which statistical significance is evaluated using Student's t-test (one-tailed or two-tailed), and the p-value is <0.05 or <0.01. Alternatively, it may refer to a state in which a substantial difference exists.

All publications and numbers identifying substances cited herein are incorporated by reference in their entirety, including structures and information linked thereto. As used herein, “or” is used when “at least one or more” of the items listed in the text may be adopted. The same applies to “or”. As used herein, a term in the format “at least one or more of A, B, and C” means that any of (A), (B), (C), (A and B), (A and C), (B and C), or (A, B, and C) can be adopted. As used herein, when “within a range of two values” is specified, the range includes the two values themselves. As used herein, “A to B” includes A and B and the values contained between A and B. As used herein, “a”, “an”, and “the” may be intended to mean that one or more elements or steps may be present, unless the context clearly indicates otherwise. As used herein, “having” includes suffering from a disease when it relates to a disease. As used herein, “the above (1) to (11)” includes a reference to any one or more of (1), (2), (3), (4), (5), (6), (7), (8), (9), (10), or (11).

While embodiments of the present invention have been described above, these are illustrative of forms that may be included in the present invention, the present invention is not limited to these, and various configurations other than those described above may also be adopted. In addition, the present invention may also adopt the respective configurations or features described in the above embodiments in combination or independently.

### EXAMPLES

The present invention will be further described below with reference to examples, but the present invention is not limited thereto.

### Experimental Methods

### Preparation of Skin Cancer Mouse Model

Male or female mice aged 8 to 12 weeks were used for the experiments. A total of 5×10⁵ B16F1 cells, which are mouse melanoma cells, were subcutaneously administered to the back of wild-type (WT) mice or CRTH2 KO (knockout) mice on a C57BL/6J background, and the tumor volume was measured. The day on which the tumor volume exceeded 100 mm³ was defined as Day 0, and Vehicle (physiological saline) or an anti-PD-1 antibody (A2122, manufactured by Selleck) was intraperitoneally administered at 5 mg/kg every 3 days, and the tumor was measured. The tumor volume was calculated by measuring the major axis and minor axis of the tumor every 3 days using a caliper (Mitutoyo, Japan) according to the following formula:

Tumor volume (mm³)=[major axis (mm)]×[minor axis (mm)]²×π/6

Experimental results are shown as mean f standard error of the mean (SEM). For significance testing, one-way analysis of variance was used, followed by Tukey's test. A p-value (P) of less than 0.05 was considered to be statistically significant.

### Example 1

### Example 1-1: Combined Administration of a CRTH2 Inhibitor and an Anti-PD-1 Antibody in a Skin Cancer Mouse Model (Wild-Type (WT))

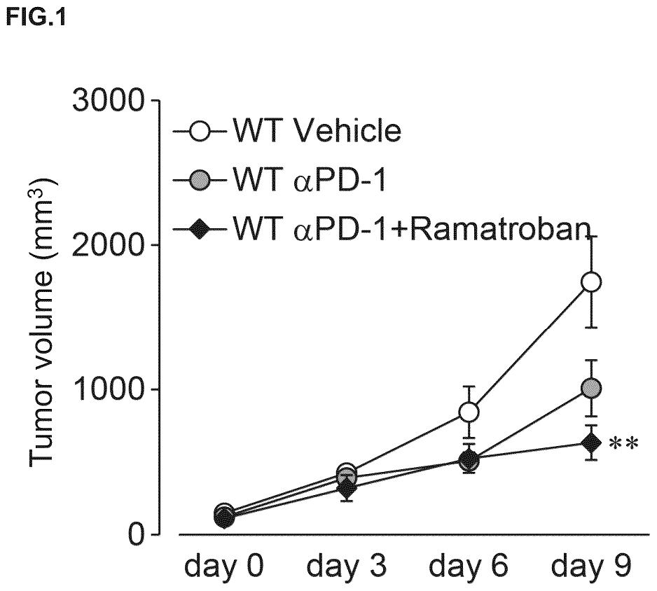

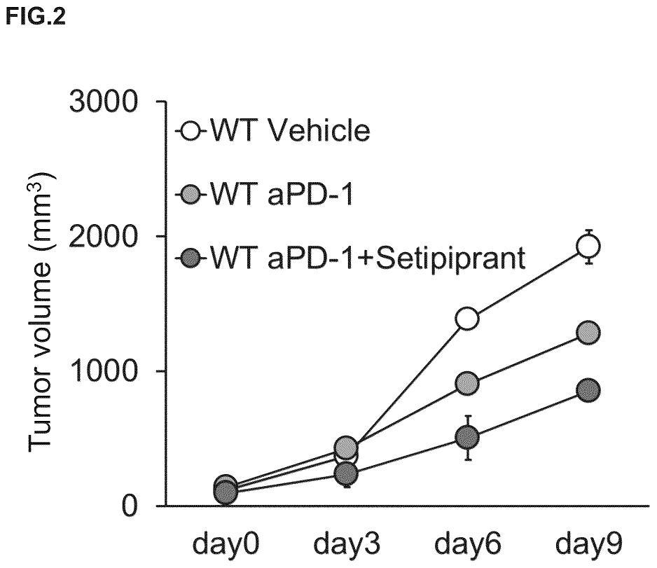

In addition to the anti-PD-1 antibody, ramatroban or setipiprant, which is a CRTH2 inhibitor, was intraperitoneally administered at 30 mg/kg three times daily from the day the cancer cells were transplanted. Tumor volumes (mm³) on Day 0, Day 3, Day 6, and Day 9 are shown in FIG. 1 and FIG. 2. Compared with the Vehicle group of the wild-type (WT), the group treated with only the anti-PD-1 antibody showed suppressed tumor growth. Furthermore, the combined treatment with the anti-PD-1 antibody and ramatroban showed additional suppression of tumor proliferation.

### Example 1-2: Administration of Anti-PD-1 Antibody to Skin Cancer Mouse Models (Wild-Type (WT) and CRTH2 KO)

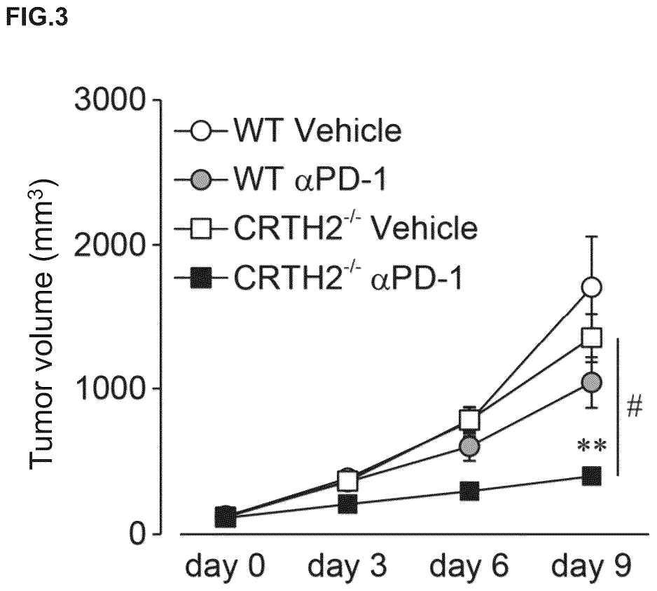

Tumor volumes (mm³) on Day 0, Day 3, Day 6, and Day 9 are shown in FIG. 3. Compared with the wild-type (WT) mice treated with the anti-PD-1 antibody, the CRTH2 KO mice showed additional suppression of tumor proliferation.

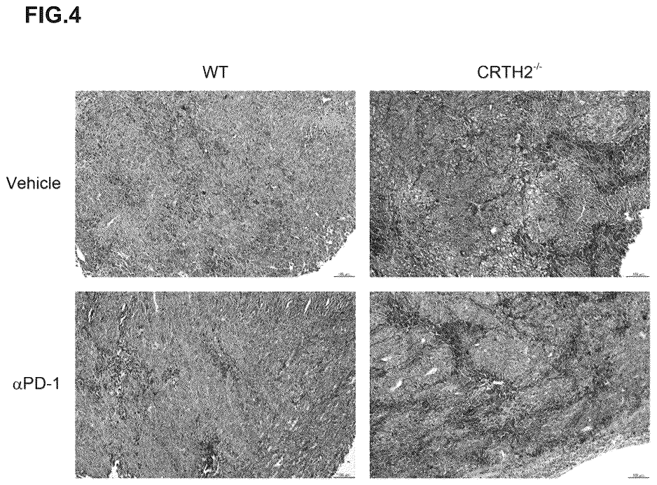

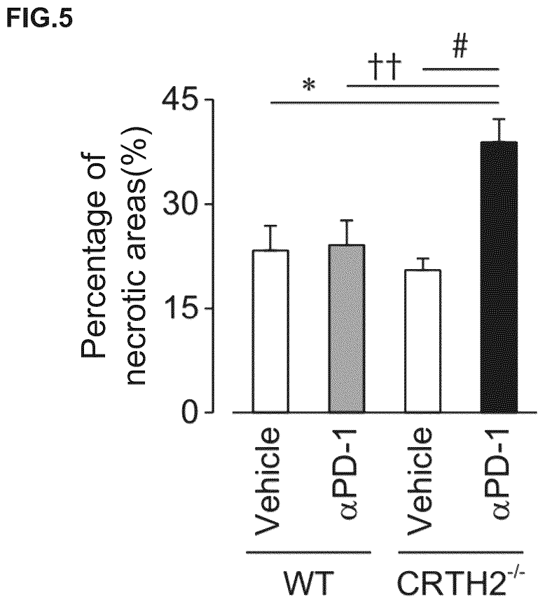

In addition to the tumor volume measurement, skin tissue was collected on day 10 and immediately immersed in 4% paraformaldehyde (4° C., 24 hours). After dehydration and clearing treatments, the tissue was embedded in paraffin, and section specimens with a thickness of 4 μm were prepared. After performing hematoxylin and eosin (H&E) staining according to a conventional method, observations were made under an optical microscope (LMD-F, Leica Microsystems, Germany), and images of the stained sections were captured using a digital camera for microscopes (K3C, Leica Microsystems, Germany). The results of H&E staining are shown in FIG. 4. It is known that cell necrosis is promoted in tumors whose proliferation is suppressed. Compared with the Vehicle group of wild-type (WT) mice, the anti-PD-1 antibody-treated group tended to show an increase in necrotic area stained light pink, and tumor cells containing black melanin granules were observed to be accumulating around the necrotic area. On the other hand, in the Vehicle group of CRTH2 KO mice, the necrotic areas were at the same level as those in the Vehicle group of wild-type (WT) mice. However, when the CRTH2 KO mice were treated with the anti-PD-1 antibody, the necrotic area further increased compared with the anti-PD-1 antibody-treated group of wild-type (WT) mice. The quantitative results are shown in FIG. 5. The percentage of necrotic areas in the entire tumor were calculated by preparing an image in which the entire tumor was filled and an image in which only the necrotic areas were filled, using Microsoft Paint. Each area was measured with ImageJ (Wayne Rasband, USA).

(Percentage of the necrotic area to the entire section)=[(Necrotic area)/(Area of the entire section)]×100

While the percentage of necrotic area in the Vehicle group of wild-type (WT) mice, the anti-PD-1 antibody-treated group of wild-type (WT) mice, and the Vehicle group of CRTH2 KO mice was approximately 20%, the percentage of necrotic area in the anti-PD-1 antibody-treated group of CRTH2 KO mice was significantly higher, approximately 40%.

### Example 1-3: Administration of Anti-CTLA-4 Antibody to Skin Cancer Mouse Models (Wild-Type (WT) and CRTH2 KO)

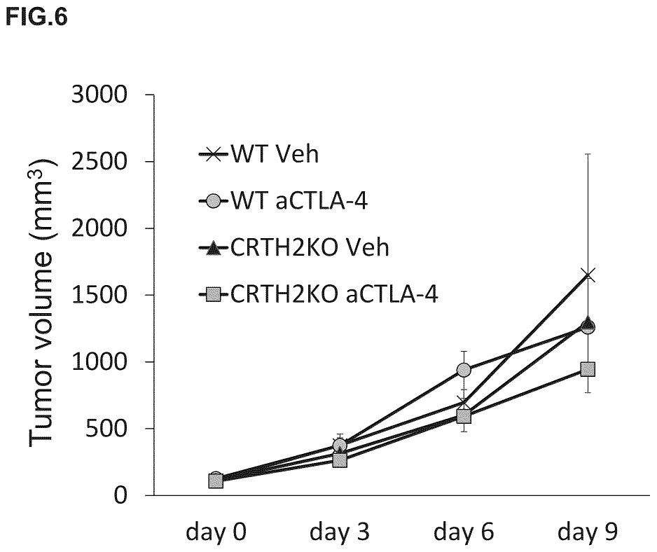

The same procedure as in Example 1-2 was performed, except that 2 mg/kg of an anti-CTLA-4 antibody was administered instead of 5 mg/kg of the anti-PD-1 antibody. Tumor volumes (mm³) on Day 0, Day 3, Day 6, and Day 9 are shown in FIG. 6. In CRTH2 KO mice, the tumor proliferation tended to be suppressed in the anti-CTLA-4 antibody-treated group compared with the Vehicle group.

### Example 1-4: Administration of Anti-PD-1 Antibody to Breast Cancer Mouse Models (Wild-Type (WT) and CRTH2 KO)

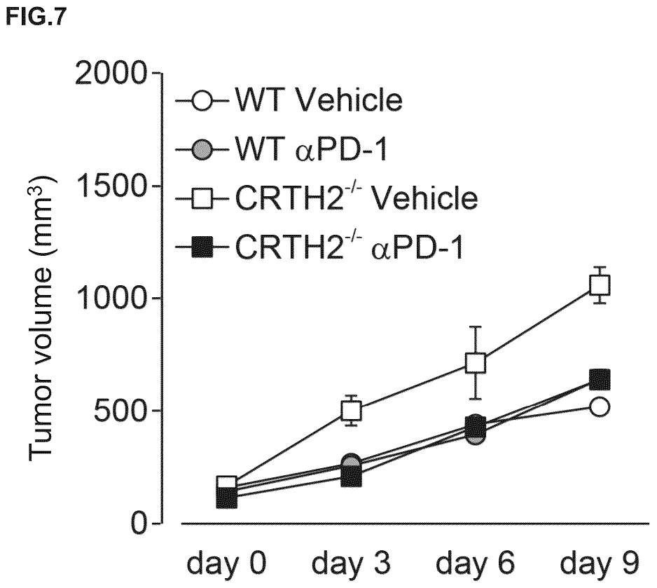

The anti-PD-1 antibody was administered to a breast cancer mouse model instead of the skin cancer mouse model. Female mice aged 8 to 12 weeks were used for the experiments. A total of 6×10⁵ 4T1 cells, which are mouse breast cancer cells, were subcutaneously administered to the fourth mammary gland of wild-type (WT) mice or CRTH2 KO mice on a BALB/c background to prepare breast cancer mouse models. Other conditions were the same as those in Example 1-2. Tumor volumes (mm³) on Day 0, Day 3, Day 6, and Day 9 are shown in FIG. 7. While there was almost no difference in tumor proliferation between the Vehicle group and the anti-PD-1 antibody-treated group of wild-type (WT) mice, the anti-PD-1 antibody-treated group of CRTH2 KO mice showed a tendency for tumor proliferation to be suppressed compared with the Vehicle group.

The results of Example 1 suggested that the anti-tumor effect of immunotherapeutic agents is enhanced by combining CRTH2 inhibition.

### Example 2

### Example 2-1: Combined Administration of a PGD2 Synthase Inhibitor and an Anti-PD-1 Antibody in a Skin Cancer Mouse Model (Wild-Type (WT))

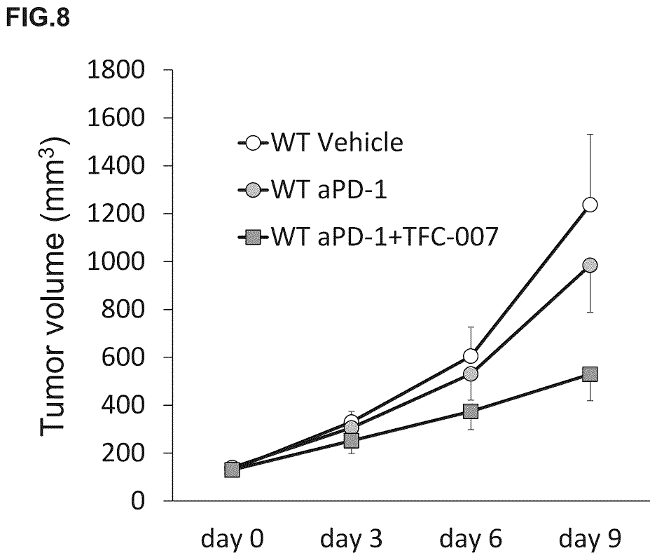

In addition to the anti-PD-1 antibody, TFC-007 (TOCRIS 5108/50), which is a PGD2 synthase inhibitor, was intraperitoneally administered at 10 mg/kg three times daily from the day the cancer cells were transplanted. The results of tumor measurement are shown in FIG. 8. Compared with the Vehicle group of the wild-type (WT), the group treated with only the anti-PD-1 antibody showed suppressed tumor growth, and the combined treatment with the anti-PD-1 antibody and TFC-007 showed a tendency to further suppress tumor growth.

### Example 2-2: Combined Administration of Anti-PD-1 Antibody to Skin Cancer Mouse Models (Wild-Type (WT) and HPGDS KO)

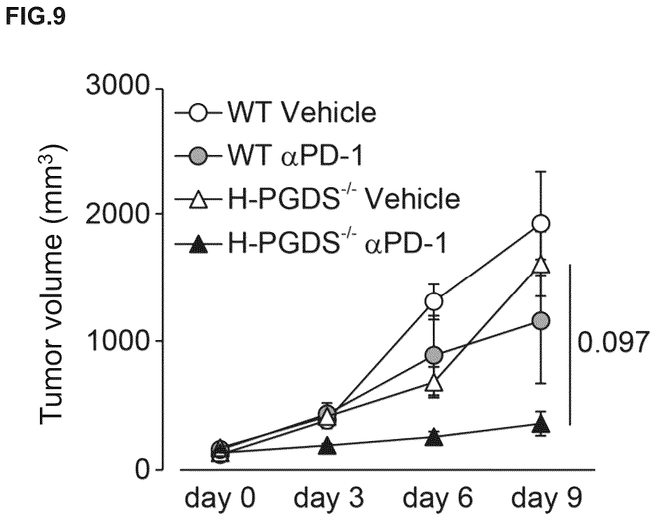

The same procedure as in Example 1-2 was performed, except that HPGDS KO mice were used instead of CRTH2 KO mice. Tumor volumes (mm³) on Day 0, Day 3, Day 6, and Day 9 are shown in FIG. 9. In HPGDSKO mice, tumor growth showed a tendency to be further suppressed compared with wild-type (WT) mice treated with anti-PD-1 antibody.

The results of Example 2 suggested that the anti-tumor effect of immunotherapeutic agents is enhanced by combining PGD2 synthase inhibition.

The present invention has been described above based on the examples. It should be understood by those skilled in the art that these examples are merely illustrative, and that various modifications are possible and such modifications are also within the scope of the present invention.

1. A method of treating a malignant tumor in a subject in need thereof, the method comprising administering to the subject an effective amount of a PGD2-CRTH2 signal inhibitor and an immunotherapeutic agent.

2. The method according to claim 1, wherein the PGD2-CRTH2 signal inhibitor and the immunotherapeutic agent are administered simultaneously.

3. The method according to claim 1, wherein the PGD2-CRTH2 signal inhibitor is administered before the immunotherapeutic agent is administered.

4. The method according to claim 1, wherein the PGD2-CRTH2 signal inhibitor is administered after the immunotherapeutic agent is administered.

5. The method according to claim 1, wherein the PGD2-CRTH2 signal inhibitor is a CRTH2 inhibitor or a PGD2 synthase inhibitor.

6. The method of claim 5, wherein the PGD2-CRTH2 signal inhibitor is:
Ramatroban;
4-(3-{3-[3-(diphenylmethyl)-6-oxopyridazin-1(6H)-yl]propyl}phenoxy)butanoic acid;
1-{6-[2-(2,4-dichloro-phenyl)-ethylamino]-2-methyl-pyrimidin-4-yl}-pyrrolidine-3-carboxylic acid;
2-(1-{2-methoxy-6-[2-(4-trifluoromethoxy-phenyl)-ethylamino]-pyrimidin-4-yl}-piperidin-3-yl)-2-methyl-propionic acid;
2-[3-{6-[2-(2,4-dichloro-phenyl)-ethylamino]-2-methoxy-pyrimidin-4-yl}-5-(1-hydroxy-1-methyl-ethyl)-phenyl]-propan-2-ol;
[6-(3-amino-piperidin-1-yl)-2-methoxy-pyrimidin-4-yl]-[2-(2,4-dichloro-phenyl)-ethyl]-amine;
[6-(4-amino-piperidin-1-yl)-2-methoxy-pyrimidin-4-yl]-[2-(2,4-dichloro-phenyl)-ethyl]-amine;
N-(1-{6-[2-(2,4-dichloro-phenyl)-ethylamino]-2-methoxy-pyrimidin-4-yl}-piperidin-4-yl)-acetamide;
5-{6-[2-(2,4-dichloro-phenyl)-ethylamino]-2-methoxy-pyrimidin-4-yl}-1-methyl-2,3-dihydro-1H-indole-2-carboxylic acid;
2-methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide;
N,N-dimethylamido-2-sulfonic acid [2-(3-{6-[2-(2,4-dichloro-phenyl)-ethylamino]-2-methoxy-pyrimidin-4-yl}-phenyl)-2-methyl-propionyl]-amide;
2-(3-{6-[2-(2,4-dichloro-phenyl)-ethylamino]-2-methoxy-pyrimidin-4-yl}-phenyl)-2-methyl-1-thiomorpholin-4-yl-propan-1-one;
2-(3-{6-[2-(2,4-dichloro-phenyl)-ethylamino]-2-methoxy-pyrimidin-4-yl}-phenyl)-isobutyramide;
2-(3-{6-[2-(2,4-dichloro-phenyl)-ethylamino]-2-methoxy-pyrimidin-4-yl}-phenyl)-N,N-dimethyl-isobutyramide;
(1-{6-[2-(2,4-dichloro-phenyl)-ethylamino]-2-methoxy-pyrimidin-4-yl}-piperidin-3-yl)-acetic acid;
1-{2-methoxy-6-[2-(4-trifluoromethoxy-phenyl)-ethylamino]-pyrimidin-4-yl}-piperidine-3-carboxylic acid;
N-(1-{2-methoxy-6-[2-(4-trifluoromethoxy-phenyl)-ethylamino]-pyrimidin-4-yl}-piperidine-3-carbonyl)-methanesulfonamide;
5-{6-[2-(2,4-dichloro-phenyl)-ethylamino]-2-methoxy-pyrimidin-4-yl}-1-methyl-2,3-dihydro-1H-indole-2-carboxylic acid ethyl ester;
(1-{6-[2-(2,4-dichloro-phenyl)-ethylamino]-2-methoxy-pyrimidin-4-yl}-piperidin-3-yl)-acetic acid ethyl ester;
N-(1-{6-[2-(2,4-dichloro-phenyl)-ethylamino]-2-methoxy-pyrimidin-4-yl}-piperidine-3-carbonyl)-methanesulfonamide;
Ethanesulfonic acid (1-{6-[2-(2,4-dichloro-phenyl)-ethylamino]-2-methoxy-pyrimidin-4-yl}-piperidine-3-carbonyl)-amide;
2-methyl-propane-2-sulfonic acid (1-{6-[2-(2,4-dichloro-phenyl)-ethylamino]-2-methoxy-pyrimidin-4-yl}-piperidine-3-carbonyl)-amide;
N-(1-{6-[2-(2,4-dichloro-phenyl)-ethylamino]-2-methoxy-pyrimidin-4-yl}-piperidine-3-carbonyl)-C, C, C-trifluoro-methanesulfonamide;
1-{6-[2-(2,4-dichloro-phenyl)-ethylamino]-2-methoxy-pyrimidin-4-yl}-piperidine-3-carboxylic acid (1H-tetrazol-5-yl)-amide;
1-{6-[2-(2,4-dichloro-phenyl)-ethylamino]-2-methoxy-pyrimidin-4-yl}-piperidine-3-carboxylic acid amide;
1-{6-[2-(2,4-dichloro-phenyl)-ethylamino]-2-methoxy-pyrimidin-4-yl}-piperidine-3-carboxylic acid dimethylamide;
N,N-dimethylamido-2-sulfonic acid 1-{6-[2-(2,4-dichloro-phenyl)-ethylamino]-2-methoxy-pyrimidin-4-yl}-piperidine-3-carboxyamide;
5-{2-methoxy-6-[2-(4-trifluoromethoxy-phenyl)-ethylamino]-pyrimidin-4-yl}-thiophene-2-carboxylic acid;
5-{6-[2-(2,4-dichloro-phenyl)-ethylamino]-2-methoxy-pyrimidin-4-yl}-2,3-dihydro-benzofuran-2-carboxylic acid;
2-(3-{6-[2-(2,4-dichloro-phenyl)-ethylamino]-2-methoxy-pyrimidin-4-yl}-phenyl)-2-methyl-propionic acid;
4-{cyclopropyl[cis,cis-4-{[4-(trifluoromethoxy)phenyl]carbonyl}-2,3,3a,4,9,9a-hexahydro-1H-cyclopenta[b]quinolin-9-yl]amino}-4-oxobutanoic acid;
4-[cyclopropyl[cis,cis-7-fluoro-2,3,3a,4,9,9a-hexahydro-4-[4-(trifluoromethoxy)benzoyl]-1H-cyclopenta[b]quinolin-9-yl]amino]-4-oxobutanoic acid;
4-[[cis,cis-6-chloro-7-fluoro-2,3,3a,4,9,9a-hexahydro-4-[4-(trifluoromethoxy)benzoyl]-1H-cyclopenta[b]quinolin-9-yl]cyclopropylamino]-4-oxobutanoic acid;
4-[cyclopropyl[cis,cis-5-fluoro-1,2,2a,3,8,8a-hexahydro-3-[4-(trifluoromethoxy)benzoyl]cyclobuta[b]quinolin-8-yl]amino]-4-oxobutanoic acid;
4-[cyclopropyl[cis,cis-6-fluoro-1,2,2a,3,8,8a-hexahydro-3-[4-(trifluoromethoxy)benzoyl]cyclobuta[b]quinolin-8-yl]amino]-4-oxobutanoic acid;
(cis,cis)-8-[(3-carboxy-1-oxopropyl)cyclopropylamino]-5-chloro-2,2a,8,8a-tetrahydrocyclobuta[b]quinoline-3(1H)-carboxylic acid 3-(phenylmethyl);
(cis,cis)-8-[(3-carboxy-1-oxopropyl)cyclopropylamino]-2,2a,8,8a-tetrahydrocyclobuta[b]quinoline-3(1H)-carboxylic acid 3-(phenylmethyl);
4-[cyclopropyl[cis,cis-1,2,2a,3,8,8a-hexahydro-3-[4-(trifluoromethoxy)benzoyl]cyclobuta[b]quinolin-8-yl]amino]-4-oxobutanoic acid;
[[[cyclopropyl[cis,cis-3-(3,4-difluorobenzoyl)-1,2,2a,3,8,8a-hexahydrocyclobuta[b]quinolin-8-yl]amino]carbonyl]oxy]acetic acid;
cis,cis-8-[(3-carboxypropyl)cyclopropylamino]-5-fluoro-2,2a,8,8a-tetrahydrocyclobuta[b]quinoline-3(1H)-carboxylic acid 3-(phenylmethyl); 4-[cyclopropyl[cis,cis-2,3,3a,4,9,9a-hexahydro-4-[4-(trifluoromethoxy)benzoyl]-1H-cyclopenta[b]quinolin-9-yl]amino]butanoic acid;
4-[cyclopropyl[cis,cis-2,3,3a,4,9,9a-hexahydro-4-[4-1[(trifluoromethyl)thio]benzoyl]-1H-cyclopenta[b]quinolin-9-yl]amino]-4-oxobutanoic acid;
(R)-1-((cis,cis-3-(benzyloxycarbonyl)-5,6-difluoro-1,2,2a,3,8,8a-hexahydrocyclobuta[b]quinolin-8-yl)(cyclopropyl)carbamoyl)azetidine-2-carboxylic acid;
4-(cyclopropyl(cis,cis-3-(4-((trifluoromethyl)thio)benzoyl)-1,2,2a,3,8,8a-hexahydrocyclobuta[b]quinolin-8-yl)amino)-4-oxobutanoic acid;
4-(ethyl(cis,cis-6-fluoro-3-(4-((trifluoromethyl)thio)benzoyl)-1,2,2a,3,8,8a-hexahydrocyclobuta[b]quinolin-8-yl)amino)-4-oxobutanoic acid;
4-(ethyl(cis,cis-7-fluoro-4-(4-((trifluoromethyl)thio)benzoyl)-2,3,3a,4,9,9a-hexahydro-1H-cyclopenta[b]quinolin-9-yl)amino)-4-oxobutanoic acid;
4-(cyclopropyl(cis,cis-7-fluoro-4-(4-((trifluoromethyl)thio)benzoyl)-2,3,3a,4,9,9a-hexahydro-1H-cyclopenta[b]quinolin-9-yl)amino)-4-oxobutanoic acid;
4-(cyclopropyl(cis,cis-7-fluoro-4-(4-(trifluoromethoxy)benzoyl)-2,3,3a,4,9,9a-hexahydro-1H-cyclopenta[b]quinolin-9-yl)amino)butanoic acid;
{3-[1-(4-chloro-phenyl)-ethyl]-5-fluoro-2-methyl-indol-1-yl}-acetic acid;
{5-fluoro-2-methyl-3-[1-(4-trifluoromethyl-phenyl)-ethyl]-indol-1-yl}-acetic acid;
{3-[1-(4-tert-butyl-phenyl)-ethyl]-5-fluoro-2-methyl-indol-1-yl}-acetic acid; {5-fluoro-3-[1-(4-methanesulfonyl-phenyl)-ethyl]-2-methyl-indol-1-yl}-acetic acid;
[5-fluoro-2-methyl-3-(1-naphthalen-2-yl-ethyl)-indol-1-yl]-acetic acid;
(5-fluoro-2-methyl-3-quinolin-2-ylmethyl-indol-1-yl)-acetic acid;
(5-fluoro-2-methyl-3-naphthalen-2-ylmethyl-indol-1-yl)-acetic acid;
[5-fluoro-3-(8-hydroxy-quinolin-2-ylmethyl)-2-methyl-indol-1-yl]-acetic acid;
(5-fluoro-2-methyl-3-quinoxalin-2-ylmethyl-indol-1-yl)-acetic acid;
[5-fluoro-3-(4-methoxy-benzyl)-2-methyl-indol-1-yl]-acetic acid;
(5-fluoro-2-methyl-3-thiazol-2-ylmethyl-indol-1-yl)-acetic acid ethyl ester;
[3-(4-chloro-benzyl)-5-fluoro-2-methyl-indol-1-yl]-acetic acid;
[5-fluoro-2-methyl-3-(4-trifluoromethyl-benzyl)-indol-1-yl]-acetic acid;
[5-fluoro-2-methyl-3-(4-tert-butyl-benzyl)-indol-1-yl]-acetic acid;
(3-biphenyl-4-ylmethyl-5-fluoro-2-methyl-indol-1-yl)-acetic acid;
[5-fluoro-3-(4-methanesulfonyl-benzyl)-2-methyl-indol-1-yl]-acetic acid;
[5-fluoro-3-(6-fluoro-quinolin-2-ylmethyl)-2-methyl-indol-1-yl]-acetic acid;
(2-methyl-3-quinolin-2-ylmethyl-indol-1-yl)-acetic acid;
(5-chloro-2-methyl-3-quinolin-2-ylmethyl-indol-1-yl)-acetic acid;
{2-[4-chloro-3-(2,6-dichloro-benzylsulfamoyl)-phenyl]-1H-indol-3-yl}-acetic acid;
{2-[4-chloro-3-(3,5-dichloro-benzylsulfamoyl)-phenyl]-1H-indol-3-yl}-acetic acid;
(2-{4-chloro-3-[2-(2,4-dichloro-phenyl)-ethylsulfamoyl]-phenyl}-1H-indol-3-yl)-acetic acid;
(2-{4-chloro-3-[2-(2-methoxy-phenyl)-ethylsulfamoyl]-phenyl}-1H-indol-3-yl)-acetic acid;
(2-{4-chloro-3-[2-(3-methoxy-phenyl)-ethylsulfamoyl]-phenyl}-1H-indol-3-yl)-acetic acid;
(2-{4-chloro-3-[2-(4-methoxy-phenyl)-ethylsulfamoyl]-phenyl}-1H-indol-3-yl)-acetic acid;
(2-{4-chloro-3-[2-(2-trifluoromethoxy-phenyl)-ethylsulfamoyl]-phenyl}-1H-indol-3-yl)-acetic acid; [2-(4-chloro-3-phenethylsulfamoyl-phenyl)-1H-indol-3-yl]-acetic acid;
{2-[4-chloro-3-(3-phenyl-propylsulfamoyl)-phenyl]-1H-indol-3-yl}-acetic acid;
2-[2-(4-chloro-3-cyclohexylsulfamoyl-phenyl)-1H-indol-3-yl]-N-methyl-acetamide;
[4-chloro-2-(4-chloro-3-cyclohexylsulfamoyl-phenyl)-1H-indol-3-yl]-acetic acid;
[4-chloro-2-(4-chloro-3-cyclohexylsulfamoyl-phenyl)-1H-indol-3-yl]-acetic acid potassium;
[2-(4-chloro-3-cyclohexylsulfamoyl-phenyl)-4-fluoro-1H-indol-3-yl]-acetic acid;
[2-(4-chloro-3-cyclohexylsulfamoyl-phenyl)-4-methyl-1H-indol-3-yl]-acetic acid;
[7-chloro-2-(4-chloro-3-cyclohexylsulfamoyl-phenyl)-1H-indol-3-yl]-acetic acid;
2-chloro-N-cyclohexyl-5-[3-(2-methanesulfonylamino-2-oxo-ethyl)-1H-indol-2-yl]-benzenesulfonamide;
2-chloro-N-cyclohexyl-5-[3-(2-ethanesulfonylamino-2-oxo-ethyl)-1H-indol-2-yl]-benzenesulfonamide;
2-chloro-N-cyclohexyl-5-[3-(2-oxo-2-trifluoromethanesulfonylamino-ethyl)-1H-indol-2-yl]-benzenesulfonamide;
2-[2-(4-chloro-3-cyclohexylsulfamoyl-phenyl)-1H-indol-3-yl]-N-(1H-tetrazol-5-yl)-acetamide;
[2-(3-cyclohexylsulfamoyl-4-ethyl-phenyl)-1H-indol-3-yl]-acetic acid;
2-[2-(4-chloro-3-cyclohexylsulfamoyl-phenyl)-1H-indol-3-yl]-propionic acid;
{2-[4-chloro-3-(3-chloro-phenylmethanesulfonyl)-phenyl]-1H-indol-3-yl}-acetic acid;
{2-[4-chloro-3-(3-chloro-phenylmethanesulfonylamino)-phenyl]-1H-indol-3-yl}-acetic acid; 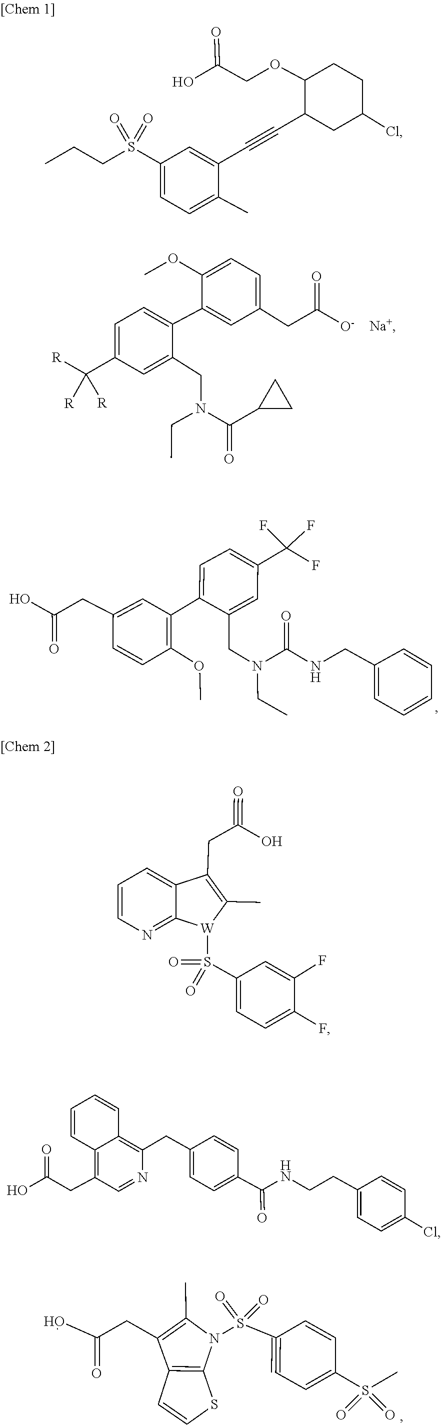 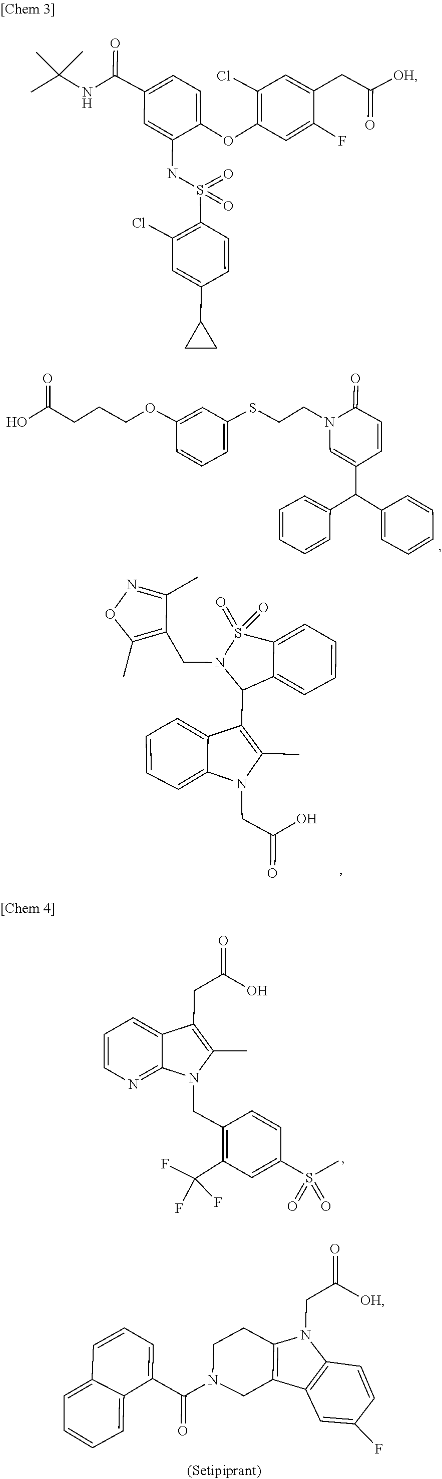 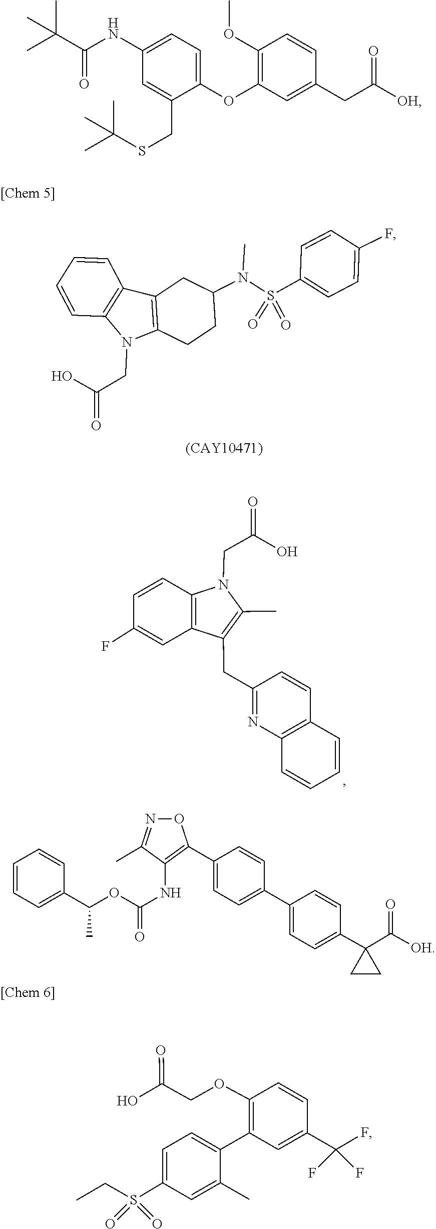 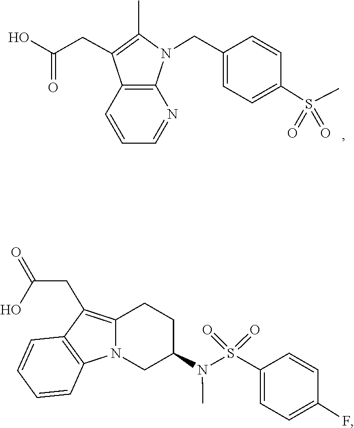 [SMILES: Cc1c(CC(=O)O)c2cccnc2n1Cc1ccc(S(C)(=O)=O)cc1 ; CN([C@@H]1CCc2c(CC(=O)O)c3ccccc3n2C1)S(=O)(=O)c1ccc(F)cc1] 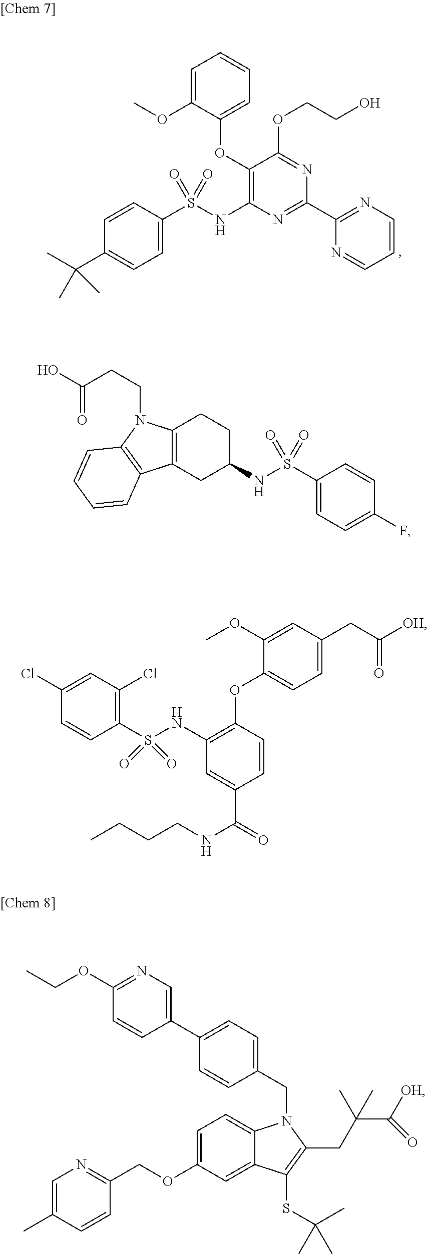 [SMILES: COc1ccccc1Oc1c(NS(=O)(=O)c2ccc(C(C)(C)C)cc2)nc(-c2ncccn2)nc1OCCO ; O=C(O)CCn1c2c(c3ccccc31)C[C@H](NS(=O)(=O)c1ccc(F)cc1)CC2 ; CCCCNC(=O)c1ccc(Oc2ccc(CC(=O)O)cc2OC)c(NS(=O)(=O)c2ccc(Cl)cc2Cl)c1 ; CCOc1ccc(-c2ccc(Cn3c(CC(C)(C)C(=O)O)c(SC(C)(C)C)c4cc(OCc5ccc(C)cn5)ccc43)cc2)cn1] 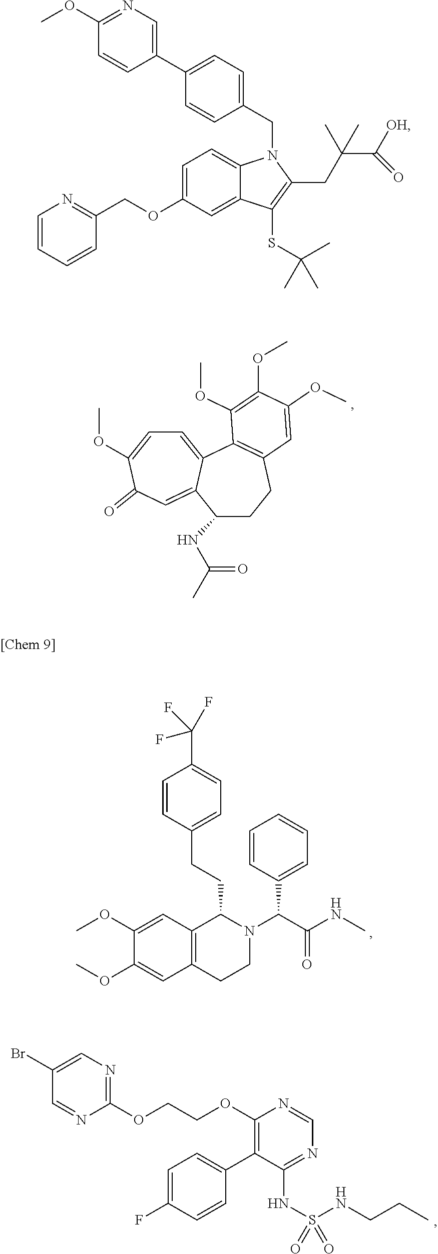 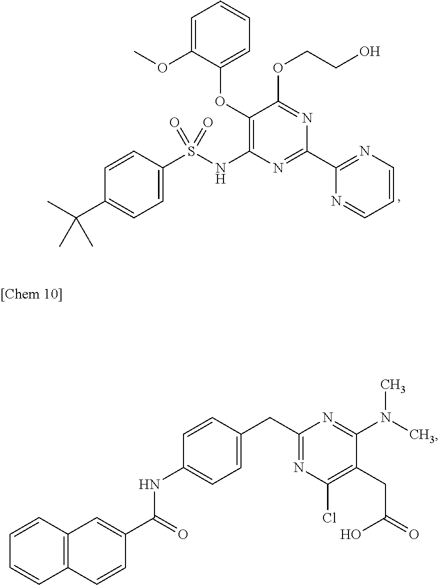 [SMILES: COc1ccccc1Oc1c(NS(=O)(=O)c2ccc(C(C)(C)C)cc2)nc(-c2ncccn2)nc1OCCO ; CN(C)c1nc(Cc2ccc(NC(=O)c3ccc4ccccc4c3)cc2)nc(Cl)c1CC(=O)O]
TFC-007, HQL-79, tranilast, pizuglanstat, AT-56, or a pharmaceutically acceptable salt thereof or a solvate thereof, or
an antibody or a PROTAC against CRTH2, H-PGDS, or L-PGDS, or
an siRNA targeting CRTH2 mRNA, H-PGDS mRNA, or L-PGDS mRNA.

7. The method of claim 5, wherein the PGD2-CRTH2 signal inhibitor is ramatroban, setipiprant, TFC-007, HQL-79, tranilast, pizuglanstat, AT-56, or a pharmaceutically acceptable salt or a solvate thereof.

8. The method according to claim 1, wherein the malignant tumor is a solid tumor.

9. The method according to claim 8, wherein the malignant tumor is lung cancer, uterine sarcoma, prostate cancer, head and neck cancer, colorectal cancer, gastric cancer, breast cancer, skin cancer, bladder cancer, kidney cancer, liver cancer, uterine cancer, pancreatic cancer, giant cell tumor of bone, ovarian cancer, esophageal cancer, adrenal cancer, biliary tract cancer, small intestinal cancer, ureteral cancer, renal pelvic cancer, penile cancer, testicular cancer, brain tumor, cancer of the central nervous system, cancer of the peripheral nervous system, thyroid cancer, salivary gland cancer, or carcinoma.

10. The method according to claim 8, wherein the malignant tumor is skin cancer or breast cancer.

11. The method according to claim 1, wherein the immunotherapeutic agent is an immune checkpoint inhibitor.

12. The method according to claim 11, wherein the immune checkpoint inhibitor is an antibody that specifically binds to an immune checkpoint molecule or a ligand thereof.

13. The method according to claim 11, wherein the immune checkpoint inhibitor is an anti-PD-1 antibody, an anti-PD-L1 antibody, or an anti-CTLA-4 antibody.

14. The method according to claim 11, wherein the immune checkpoint inhibitor is a PD-1 inhibitor, a PD-L1 inhibitor, or a CTLA-4 inhibitor.

15. The method according to claim 11, wherein the immune checkpoint inhibitor is nivolumab, pembrolizumab, atezolizumab, durvalumab, or ipilimumab.
